(19) United States
(12) Patent Application Publication
(10) Pub. No.: US 2026/0294772 A1
(43) Pub. Date: Oct. 1, 2026

(54) COMPLEX COMBINING A HETEROCYCLIC NITROGEN BIOMOLECULE WITH A SILICIC ACID, AND APPLICATIONS

(71) Applicant: EXSYMOL, Monaco (MC)

(72) Inventor: Marco CAPPONI, Imperia (IT)

(73) Assignee: EXSYMOL, Monaco (MC)

(21) Appl. No.: 19/574,929

(22) Filed: Mar. 23, 2026

(30) Foreign Application Priority Data
Apr. 1, 2025 (FR) 2503392

(51) Int. Cl. A61K 8/60; A61K 8/25; A61K 8/49; A61Q 7/00; A61Q 19/00; A61Q 19/08

(52) U.S. Cl. CPC A61K 8/606; A61K 8/25; A61K 8/4953; A61Q 7/00; A61Q 19/008; A61Q 19/08; A61K 2800/58

(57) ABSTRACT

The present invention relates to new complexes formed between a) a heterocyclic nitrogen biomolecule and b) a silicic acid, compositions comprising them, and their applications.

### CROSS-REFERENCE TO RELATED APPLICATIONS

This application claims priority to French Patent Application No. FR2503392, filed Apr. 1, 2025, which is incorporated by reference in its entirety.

### FIELD OF THE INVENTION

The present invention relates to new complexes combining a heterocyclic nitrogen biomolecule with a silicic acid, compositions comprising them, and their applications.

### STATE OF THE ART

Nucleic acids, including the two major forms, deoxyribonucleic acid (DNA) and ribonucleic acid (RNA), are natural macromolecules found universally in all living organisms. In addition to their widely recognized uniqueness as the hereditary material responsible for storing, encoding and transferring genetic information to other cellular components, they also play a fundamental role in humans through their involvement in numerous biological processes within the body, such as catalyzing certain biochemical reactions and regulating certain activities in cells, etc (Wang F. et al., Biosensors, 2022, vol. 12, pp. 1-35 and references cited). As stated in the online guide to cosmetic ingredients for premium beauty formulations, nucleic acids nowadays represent a group of bioactive molecules and ingredients that are considered “cutting-edge” because they are innovative, scientifically proven, effective and most often incorporated into exclusive skincare products (https://inci.guide/nucleic-acids). As a result, in cosmetics, dermatology and even in the fields of aesthetic and regenerative medicine, these nucleic acids, as well as their fragments such as polynucleotides and polydeoxyribonucleotides (linear biopolymers often described, for convenience, using the respective and common abbreviations “PN” and “PDRN”), and even their simple basic constitutive monomeric units, have seen their use grow in popularity and potential in recent years when it comes to skin renewal and bio-restructuring (Zakrewsky M. et al., J. Control Release, 2015, vol. 219, pp. 445-456). Therefore, as a first illustration of the action and benefits which the aforementioned “nucleic acids and resulting fragments” have on the skin, an article published this year reports promising results for the aforementioned PNs in terms of reduction in wrinkle depth, their ability to improve skin texture, etc, and, beyond that, concludes that they are an effective and well-tolerated cosmetic treatment (Lampridou S. et al. J. Cosmet. Dermatol., 2025, vol. 24, e16721). A few months earlier, another study conducted on these same PNs reported significant improvements in skin elasticity and hydration (Lee K. W. A. et al., Int. J. Mol. Sc., 2024, vol. 25, 8224). As for the aforementioned PDRNs, which are natural fragments of DNA of low molecular weight ranging from 50 to 1,500 kDa (Marques C. et al., Biomolecules, 2025, vol. 15, 148), the state of the art confers on them a wide range of skin benefits, ranging from promoting cell activity and collagen synthesis to treating hyperpigmentation, revitalizing the skin, angiogenesis, etc (Khan A. et al., Chinese J. Plastic Rec. Sur., 2022, vol. 4, pp. 187-193), which moreover led the same team of authors to choose an evocative title for their article, namely: “Polydeoxyribonucleotide: a promising skin anti-aging agent”. In accordance with the above observation, these same PDRNs are also known for their ability to stimulate neocollagenesis in photoaged skin, as well as their potential for skin whitening due to their depigmenting properties (Noh T. K. et al., Int. J. mol. Sc., 2016, vol. 17, 1448). Furthermore, as a second illustration, focusing this time on the action and benefits for the skin of the aforesaid “basic monomeric constitutive units of nucleic acids”, in other words the five purine and pyrimidine nitrogenous bases, namely adenine, guanine, cytosine, thymine, and uracil, the state of the art displays, for example, such as for adenine which is a purine nucleic base, an ability to delay the senescence of cells located in the papillary dermis, a thin area located at the dermo-epidermal junction of the skin (Cheng Y-F. et al., Exp. Dermatol., 2016, vol. 25, pp. 151-164). The same adenine, albeit functionalized with the compound known as N⁶-(4-hydroxybenzyl)adenine riboside, has itself displayed, on a mouse model of ageing, benefits against the cellular senescence induced by different typologies of oxidative stress, and therefore demonstrates the potential to be a powerful agent for preventing age-related neurodegeneration and neuroinflammation (Hsu W. H. et al., Aging, 2020, vol. 12(11), pp. 10556-10577). For its part, and for cosmetic and dermatological applications, guanine, another purine nucleic base, is considered, when combined with an aldopentose, to be an active ingredient useful in maintaining skin homeostasis, particularly in the prevention and/or treatment of skin irritation (patent FR2975595). It should also be noted that nucleic acid in its pre-eminent form (DNA) is composed of four of the five nitrogenous bases listed above, and that each of these four nucleobases (adenine, guanine, cytosine and thymine) is listed in the International Cosmetic Ingredient Dictionary and Handbook published by the Personal Care Products Council (PCPC) of the American Cosmetic Association, in other words, the reference standard used worldwide by the cosmetics industry to standardize and normalize the names of all ingredients present in commercially available skincare product formulas. In any case, and at this stage of the description of the present invention, it should be noted that the list of skin benefits associated with the use of nucleic acids and derivatives as active and functional ingredients on the skin is now extensive and promising, with a large number of studies on the subject that have been published over the last decade. Within the meaning of the present application, the aforementioned “nucleic acids and derivatives” should be understood using a holistic approach, i.e. encompassing both an organic macromolecule (polymer) consisting of a succession of nucleotides and its constituent monomeric units (the latter being either free or functionalized by an N-glycosidic bond). However, despite this considerable effort to better understand the skin targets and mechanisms of action of these nucleic acids and derivatives on skin, several limitations and obstacles still remain to their use and must be considered on a case-by-case basis for an expected optimal use in cosmetics and dermocosmetics. In this regard, one issue that can be cited is the stability of these nucleic acids and derivatives, particularly when applied topically to the skin, due to environmental factors (ultraviolet radiation, heat, humidity), biological factors (nucleases and natural skin enzymes) and chemical factors (pollution, pH variations, interaction with other ingredients), all of which are likely to cause degradation and, consequently, a loss of efficacy (Ingle R. G. et al., Pharmaceutics, 2023, vol. 15, 1158; Probst J. et al., Genetic Vaccines and Therapy, 2006, vol. 4(4), pp. 1-9). As previously indicated, this proves particularly problematic regarding the topical application/administration of these nucleic acids and derivatives to the skin. Indeed, since the stratum corneum of the skin's epidermis acts as a protective barrier, the cutaneous bioavailability and ability of these same nucleic acids and derivatives to penetrate the epidermis in order to exert a targeted action at the cellular level are also likely to be affected, or even display a very limited diffusion when deeper layers of the skin are targeted (Zakrewsky M. et al., J. Control Release, 2015, vol. 219, pp. 445-456 and references cited). Similarly as a “challenge” to overcome with a view to optimizing the use of the said nucleic acids and derivatives in cosmetics and dermocosmetics, particularly in the context of topical skin application, there is their physicochemical behavior in solution. Indeed, PDRNs and PNs do not dissolve easily in water and generally need to be subjected to heat treatment, which can therefore degrade their characteristics and qualities (Marques C. et al., Biomolecules, 2025, vol. 15, 148). As for the aforementioned nucleobases (also referred—more simply—as “bases”), their potential for application is generally also hampered by their low solubility in water (Ghoshdastidar D. et al., J. Phys. Chem. B., 2016, vol. 120(3), pp. 492-503). Regarding strategies to overcome any one of these limitations on the use of nucleic acids and derivatives, several concepts of general action have been reported (Wang F. et al., Biosensors, 2022, vol. 12, pp. 1-35 and references cited). By way of illustration, we can mention, for example, their integration into different types of nanomaterials, such as lipid-based nanoparticles or inorganic nanoparticles, in order to achieve superior performance in the targeted biomedical applications, particularly in the treatment of skin wrinkles (Yeo S. et al., Pharmaceutics, 2021, vol. 13, 33), or even mention their combination with a panel of hydrotropic agents bearing an amphiphilic molecular structure (patent US2014107059). The state of the art also displays chemical modifications that are made to natural nucleic acids with the aim of improving their biological behavior, such as a class of biocompatible chemical reactions (collectively and expressively referred to as “click chemistry”) which seeks to “hook” specific bioactive and functional molecules to them by conjugation, ultimately resulting—according to the authors of the said article—in “nucleoside hybrids” (Perrone D. et al., Molecules, 2021, vol. 26, 3100). Furthermore, and due to its affinity for heteroaromatic compounds, arginine may thus be able to increase the solubility of all nucleobases tested, provided that these nucleobases are electrically neutral (Hirano A., Arch. Biochem. Biophys., 2010, vol. 497(1-2), pp. 90-96). Finally, emphasis is placed on the more distant approach aiming at amplifying the solubility of substrates known to be poorly soluble in water (the latter being first illustrated in the study by the nucleobase “adenine”) by, and with the determination of apparent association constants (Ka), the formation and obtaining of “complex” entities resulting from an association between the said substrate that is poorly soluble in water and a selection of appropriate ligands such as water-soluble vitamins and amino acids (Chen A. X. et al., Pharm. Res., 1994, vol. 111(3), pp. 398-401).

In any case, despite the availability of various solutions intended to improve the cutaneous administration of nucleic acids and derivatives, it is important to note that other studies conversely report limited or even no benefits (Lee K. W. A. et al., Int. J. Mol. Sc., 2024, vol. 25, 8224). At the same time, there is still a need to continue to further pursue research aimed at establishing the most optimal conditions of use and efficacy for a more universal application of nucleic acids and derivatives in cosmetics and dermocosmetics, in particular by evaluating, as for the aforementioned PDRNs, their function and efficacy in eclectic bioactivities/indications (Nguyen T. H. et al., Int. J. Biol. Macromolecules, 2024, vol. 282(3), 137051). Thus, the present invention fully addresses such a challenge and the need to design and make available [particularly on the market for cosmetic and/or dermocosmetic active ingredients categorized as “nucleic acids and derivatives” ] a new ingredient that meets all or part of the above-mentioned needs and aims to achieve all or part of the following objectives:
i) to address the aforementioned limits of use for nucleic acids and derivatives: stability, bioavailability and solubility;
ii) to maximize the biological potential of nucleic acids and their derivatives, in particular to prevent and/or combat cosmetic and dermocosmetic disorders that may affect both the physiology of the skin of the body and, more specifically, that of the scalp;
iii) optionally (but nevertheless advantageously) to display an additional component to prerequisites i) and ii), namely to exhibit a total naturalness for said ingredient, in essence to present a naturalness index of 100% within the meaning of the international standard ISO 16128 [text aimed at harmonising and regulating the practices of players in the cosmetics industry with regard to what is “natural”, in particular providing guidelines on the definitions and criteria applicable to natural and organic cosmetic ingredients and products, especially establishing a method for determining the “Natural”, “Of Natural Origin”, “Organic” and “Of Organic Origin” indexes applicable to cosmetic ingredients—Reference number: ISO 16128-2:2017(F)].

Regarding this (optional) requirement iii) above, its origin arises from a strong trend and demand currently observed among cosmetics formulators, namely that, in a world that is fortunately increasingly concerned about environmental issues and sustainability, the cosmetics industry is also evolving by seeking to gradually integrate more responsible and environmentally friendly practices. As a result, key concepts such as “naturalness”, “circular economy”, “eco-design” and “green cosmetics” have emerged in recent years, as reported in the white paper “Circular Economy and the Cosmetics Sector—120 best practices for cosmetics companies” published by the Federation of Beauty Companies (FEBEA—Fédération des Entreprises de la Beauté), the French trade association for cosmetics manufacturers. In essence, this white paper addresses the challenges and opportunities now facing the cosmetics sector in the face of the need to reduce the environmental impact of cosmetics production. It highlights concrete solutions to encourage waste reduction, resource optimization and material reuse, while ensuring that product quality and safety are maintained. Through detailed recommendations, this compilation of best practices underlines the need to adopt a more responsible approach throughout the product life cycle, from design to end of life. In general, this white paper invites all players in the cosmetics sector to radically rethink their production methods, in particular by promoting a circular economy and a so-called “industrial” ecology with a focus on minimizing waste, maximizing the use of resources, implementing effective recycling systems or even creating sustainable inter-company ecosystems, such as the (sic) ““Waste for some, raw materials for others.”” found on the web. Located at the beginning of the product supply chain, suppliers of cosmetic ingredients are all the more confronted with the challenge of innovating in the context of the four key concepts mentioned above, as there is a growing and even immediate need for alternatives—to conventional fossil-based ingredients—that are sustainable, effective and respectful of the environment and biodiversity (Goyal N., et al., Env. Sc. Pollution Res., 2023, vol. 30, pp. 25148-25169). The applicant and its inventors therefore also had the ambition to seel to innovate while taking such an observation into account.

### DESCRIPTION OF THE INVENTION

The present invention was therefore developed in the context of the—ambitious—research and specifications such as indicated above, involving the design and implementation of a panel of tests aimed at establishing and developing biological evaluation models, both for skin and hair, capable of demonstrating the efficacy of, and highlighting the benefits of, the ingredients tested. Thus, after extensive research, and moreover contrary to all expectations given the admitted knowledge that orthosilicic acid Si(OH)₄ (or H₄SiO₄), the natural form of silicon that can be assimilated by humans, has the disadvantage of being present in low concentrations in water (<2 mM or 56 mg Si/l) due to a very strong tendency to self-condense as soon as silicon levels rise, and consequently to adversely produce polymerized forms that are poorly absorbable, or more difficult to absorb, by the body and which are therefore potentially inert or biologically inactive (Jurkić L. M. et al., Nutr. Meta., 2013, vol. 10, pp. 1-12), the applicant has succeeded in designing, in compliance with controlled ratio and conditions, a new molecular complex (also known as a “supramolecular” complex) combining, through interaction (supramolecular chemistry), on the one hand, a limited family of nucleobases and derivatives with, on the other hand, a form (preferably natural) of soluble, bioavailable, biologically assimilable silicon, which is moreover [in accordance with the optional prerequisite referred to in point iii) above] advantageously derived initially from a by-product (waste) generated by the cereal industry, and with, in addition, a broad and advantageous general behavior in cosmetics and dermocosmetics [in accordance with the prerequisite referred to in point ii) above]. Thus, the invention relates to a (molecular) complex formed between:
a) a heterocyclic nitrogen biomolecule selected from purine nucleobases and/or their methylxanthine alkaloid derivatives, free or in N-glycosylated form; and
b) a silicic acid consisting of orthosilicic acid and/or at least one of its water-soluble oligomers;
   said complex being formed via the establishment of at least one weak chemical bond, preferably at least one hydrogen bond, between at least one hydroxyl group of said silicic acid b) and said heterocyclic nitrogen biomolecule a), preferably at the level of at least one of the nitrogen atoms of said heterocyclic nitrogen biomolecule a); the molar ratio between a) and b) being between about 0.5 (e.g. 0.5) and about 15 (e.g. 15).

According to one embodiment, the complex according to the invention is formed via the establishment of weak bonds, preferably hydrogen bonds, between the hydroxyl groups of said silicic acid b) and the nitrogen atoms of said heterocyclic nitrogen biomolecule a).

Indeed, after conducting a large number of trials on both the ratio between the constituent elements of said complex and the operating conditions, the applicant opted for such a new chemical entity and intermolecular association, in the form of a “complex” as defined by the nomenclature rules of the International Union of Pure and Applied Chemistry (“UICPA” in French for “Union Internationale de Chimie Pure et Appliquee”, or “IUPAC” in English), due to the advantageous and promising general behavior that said complex according to the invention displayed through a range of beneficial technical effects, in particular for one of said effects, namely the experimental superiority that said complex according to the invention has displayed in comparison with each of the constituent elements taken individually (to be understood as “not engaged in a same complex state”), this range of beneficial technical effects being precisely illustrated by:
its ability to significantly limit the secretion of lipids (sebum) produced and accumulated in and by sebocytes [see test 1 below];
its ability to significantly stimulate the proliferation of human hair follicle dermal papilla cells (better known by the abbreviation “HFDPC”), said papilla being located at the base of the hair follicle and therefore playing an important role in the growth phase (anagen) of the hair [see test 2 below];
its ability to significantly limit hyperproliferation of keratinocytes induced by an inflammatory stress, thus suggesting a protective effect of the complex according to the invention on said keratinocytes (constituent cells of the superficial layer of the skin known as the epidermis, as well as of appendages such as hair and nails) [see test 3 below];
finally, its ability to significantly improve the skin viscoelasticity on skin explants and through topical application, suggesting that the complex according to the invention has the ability to restore the viscoelastic behavior of ageing skin [see test 4 below].

In said test 2 below, it is emphasized that the study conducted and the complex according to the invention made it possible to demonstrate a biological behavior for the latter in a much more effective manner—with a broadly superior effect—than the first component of said complex (adenine functionalized by an N-glycosidic bond, in this case a ribofuranose group via a β-N(9)-glycosidic bond), alone, or the second component of said complex (silicic acid), alone.

Furthermore, test 2 below reveals, in a completely unexpected manner, a phenomenon of synergy action for such a complex according to the invention [presenting, in this case, in the form of a complex formed between adenine functionalised by an N-glycosidic bond, in this case a ribofuranose group via a β-N(9)-glycosidic bond, and silicic acid]; such synergy of action is, moreover, legitimately posited:
in view of the theoretical teaching provided for the use of such a phrase in the field of chemical substances and products, in particular the fact that (sic) “there is a synergistic effect when the combined effect of two chemical product is much greater than the sum of the effects of each product taken individually. For example: 2+2>>4”
[https://www.cchst.ca/oshanswers/chemicals/synergism.htm];
in light of the experimentation and results obtained in test 2 below, since the combination of the two components which are the subject of the complex according to the invention produces a much better effect than that obtained at equivalent use concentrations for each component taken individually.

It is also legitimately posited that this specific mode of action, with this objectified phenomenon of synergy of action, makes it possible to refer to the biological behavior of the aforementioned complex illustrated as “optimal” or “endowed with optimal properties” for hair growth and therefore as an active ingredient intended to promote hair growth/regrowth. Finally, it is legitimate to posit the formation and existence of a (molecular) “complex” state, in view of, on the one hand, the definitions of “complex” and “hydrogen bond” established by the International Union of Pure and Applied Chemistry (“UICPA” in French, or “IUPAC” in English), on the other hand and for purine nucleobases and derivatives that have been specifically selected, at their electron-rich regions, marked by the presence of non-bonding electron doublets and acid hydrogen atoms on certain nitrogen atoms, which are precisely conducive to the establishment of such “hydrogen bond”-type interactions with at least one hydroxyl group of silicic acid. To the applicant's knowledge, the aforementioned family of heterocyclic nitrogen biomolecules a) and the aforementioned silicic acid b) (ortho-silicic acid and/or at least one of its water-soluble oligomers) have never been combined in the form of a molecular complex, either for use in cosmetics and/or dermocosmetics, or even with regard to disorders/alterations that may affect both the physiology of the skin of the body and, more specifically, that of the scalp. Furthermore, any phenomenon of synergy action between the aforementioned family of heterocyclic nitrogen biomolecules a) selected from purine nucleobases and/or their methylxanthine alkaloid derivatives, and the aforementioned silicic acid b), whether in a “complex” state or not (simple association), has never been described or even predicted in the prior art. In addition, in accordance with the objectives attached to the technical problem posed by the present invention, the aforementioned complex according to the invention has the advantage, according to an advantageous embodiment of the invention, of displaying a total naturalness index, namely an index of 100% natural origin within the meaning of the international standard ISO 16128.

According to a particular embodiment, the invention also relates to a (molecular) complex formed between:
a) a heterocyclic nitrogen biomolecule selected from purine nucleobases and/or their methylxanthine alkaloid derivatives, free or in N-glycosylated form; and
b) a silicic acid consisting of orthosilicic acid and/or at least one of its water-soluble oligomers;
   said complex being formed via the establishment of at least one weak chemical bond, preferably at least one hydrogen bond, between at least one hydroxyl group of said silicic acid b) and said heterocyclic nitrogen biomolecule a), preferably at the level of at least one of the nitrogen atoms of said heterocyclic nitrogen biomolecule a).

According to one embodiment of the invention, said heterocyclic nitrogen biomolecule is a free purine nucleobase, preferably adenine or guanine, advantageously adenine.

According to another embodiment of the invention, said heterocyclic nitrogen biomolecule is an N-glycosylated purine nucleobase, preferably associated with D-ribose.

Advantageously, said N-glycosylated purine nucleobase is selected from 9-β-D-ribofuranosyladenine and/or 9-β-D-ribofuranosylguanine, preferably 9-β-D-ribofuranosyladenine (in IUPAC systematic nomenclature).

According to another embodiment of the invention, said heterocyclic nitrogen biomolecule is a methylxanthine alkaloid derived from a purine nucleobase, selected from 1,3,7-trimethylxanthine, 1,3-dimethylxanthine, 3,7-dimethylxanthine and/or 1,3-dimethylxanthine-7-acetic acid, preferably consisting of 1,3,7-trimethylxanthine (in systematic IUPAC nomenclature).

According to one embodiment of the invention, said silicic acid is orthosilicic acid (H₄SiO₄).

According to one embodiment of the invention, said water-soluble oligomers of orthosilicic acid are disilicic acid (H₆Si₂O₇), linear trisilicic acid (H₈Si₃O₁₀) and cyclic trisilicic acid (H₆Si₃O₉), preferably disilicic acid (H₆Si₂O₇).

According to one embodiment of the invention, said silicic acid consists of orthosilicic acid and optionally at least one of its water-soluble oligomers.

According to one embodiment of the invention, said silicic acid consists of orthosilicic acid and at least one of its water-soluble oligomers. In this embodiment, the proportion of orthosilicic acid is preferably equal to or greater than 70%, preferably equal to or greater than 90%, advantageously 100%.

In a preferred embodiment of the invention, on the one hand said heterocyclic nitrogen biomolecule a) is adenine or 9-β-D-ribofuranosyladenine, preferably 9-β-D-ribofuranosyladenine, and on the other hand said silicic acid b) is selected from orthosilicic acid and disilicic acid.

In a preferred embodiment of the invention, said silicic acid b) is derived from silicon-containing plant biomass, preferably selected from the grass species Oryza sativa (rice), Panicum miliaceum (millet), Triticum spp. (wheat), Zea mays (maize), Hordeum vulgare (barley), Avena sativa (oats), Secale cereale (rye), Sorghum bicolor (sorghum), Triticum dicoccum (spelt), Triticum turanicum (Khorasan wheat), Eragrostis tef (teff), Digitaria exilis (fonio), and the pseudo-cereals Chenopodium quinoa (quinoa), Amaranthus spp. (amaranth), Fagopyrum esculentum (buckwheat), in particular selected from the grass species Oryza sativa (rice) and Panicum miliaceum (millet), advantageously the species Oryza sativa (rice).

Preferably, said silicon-containing plant biomass is a by-product of the cereal industry, preferably the husk of Oryza sativa (rice). In this regard indeed, and in particular for the purposes of point iii) above, the applicant has turned to a raw material derived from recycling (known as “upcycling” in English), in this case the husk of Oryza sativa (rice), which is nothing more than the outer shell covering the rice grain. There were two reasons for the primary choice of this material: on the one hand, it is clearly a natural by-product of rice husking. Indeed after the grains have been extracted, these rice husks are most often considered as agricultural waste, produced in large quantities due to the very significant activity of the rice industry for the global population. Fortunately for more than a decade, these rice husks and large volumes of waste have found numerous applications in fields as varied as poultry farming, composting, combustion where they are used as biomass to be supplied to fuel reactors and thus produce thermal or electrical energy, etc (Moraes C. A. M. et al., Waste Management & Research, 2014, vol. 32(11), pp. 1034-1048). On the other hand, and of interest to the applicant in the context of its research and specifications as indicated above, it should be noted that these rice husks are also rich in silicon dioxide (SiO₂), more commonly known as “rice husk silica”, which is a sustainable alternative to traditional sources of silica for industries seeking environmental responsibility. (Islam T. et al., Asian J. Chem. Sci., 2024, vol. 14(2), pp. 83-96). Finally, it is reported that this rice husk silica, once heat-treated, is converted into amorphous silica, a precursor form and particularly interesting candidate for the delivery, up to its solubility limit, of the aforementioned orthosilicic acid (Ali M. et al., Int. J. Cur. Res. Rev., 2021, vol. 13(24), pp. 94-99).

### Advantageously:

i) the molar ratio between said heterocyclic nitrogen biomolecule a) and said silicic acid b) is between about 1 and about 7 (e.g. between 1 and 7), advantageously between about 2 and about 3 (e.g. between 2 and 3), and/or
ii) the pH of the composition (in liquid form, advantageously in the form of a solution) comprising said complex is between about 2.5 and about 4.5 (e.g. between 2.5 and 4.5), advantageously between about 3 and about 3.5 (e.g. between 3 and 3.5), advantageously about 3 (e.g. 3);
   preferably, the complex and the liquid composition comprising it (advantageously in the form of a solution) having characteristics i) and ii) as illustrated by its preparation process [see test 5 below].

The invention also relates to a composition, preferably in the form of a solution, said composition comprising (or consisting essentially of) at least one complex according to the invention, wherein:
i) the molar ratio between said heterocyclic nitrogen biomolecule a) and said silicic acid b) is between about 0.5 and about 15 (e.g. between 0.5 and 15), preferably between 1 and about 7 (e.g. between 1 and 7), advantageously between about 2 and about 3 (e.g. between 2 and 3), and/or
ii) the pH of the composition (in liquid form, advantageously in the form of a solution) comprising said complex is between about 2.5 and about 4.5 (e.g. between 2.5 and 4.5), preferably between about 3 and about 3.5 (e.g. between 3 and 3.5), advantageously about 3 (e.g. 3);
   preferably said composition having characteristics i) and ii).

Incidentally and from a semantic point of view, it should be noted that the expression “consisting essentially of” used above has the same meaning as the expression “composed essentially of”; these two formulations being interchangeable.

According to a particular embodiment, the invention relates to a composition, preferably in the form of a solution, said composition comprising (or consisting essentially of) at least one complex formed between:
a) a heterocyclic nitrogen biomolecule selected from purine nucleobases and/or their methylxanthine alkaloid derivatives, free or in N-glycosylated form (as defined above); and
b) a silicic acid consisting of ortho-silicic acid and/or at least one of its water-soluble oligomers (as defined above);
   said complex being formed via the establishment of at least one weak chemical bond, preferably at least one hydrogen bond, between at least one hydroxyl group of said silicic acid b) and said heterocyclic nitrogen biomolecule a), preferably at the level of at least one of the nitrogen atoms of said heterocyclic nitrogen biomolecule a); preferably, the pH of said composition (in liquid form, advantageously in the form of a solution) being between about 2.5 and about 4.5 (e.g. between 2.5 and 4.5), preferably between about 3 and about 3.5 (e.g. between 3 and 3.5), advantageously about 3 (e.g. 3).

The invention also relates to a cosmetic or dermocosmetic composition, characterized in that said composition comprises (or consists essentially of), in combination with at least one cosmetically or dermocosmetically acceptable excipient, at least one complex or composition according to the invention, preferably said cosmetic or dermocosmetic composition furthermore being in a form suitable for topical administration to the skin and said at least one excipient furthermore being physiologically acceptable to the skin. Advantageously, the quantity of said composition according to the invention in the aforementioned compositions is between 1% and 10% by weight relative to the total weight of said composition, preferably between 3% and 7% by weight relative to the total weight of said composition. By way of example and without limitation, when the aforementioned compositions for cosmetic or dermocosmetic use are suitable for topical administration to the skin, they may be presented in any form normally used for such administration. Thus, and advantageously, they may be in liquid form, preferably in the form of a solution (for example in the form of an aqueous or hydroalcoholic solution), possibly gelled, a dispersion such as a lotion, possibly biphasic, an oil-in-water or water-in-oil emulsion, a suspension, a lotion, a cream, an aqueous or hydroalcoholic gel, or even in the form of a (micronized) powder, a foam, a serum, a paste, a suspension, a dispersion, or multiple emulsions that may optionally be microemulsions or nanoemulsions. In terms of adjuvants present in the above-mentioned compositions and physiologically compatible with the skin, we can mention a compound chosen from oils, waxes, silicone elastomers, surfactants, co-surfactants, solubilizers, co-solubilizers, thickeners and/or gelling agents, humectants, emollients, organic or inorganic sunscreens, photostabilizers, preservatives with the exception of aldehyde-donor preservatives, colouring agents, abrasive fillers, pearlescent agents, mattifying agents, tightening agents, sequestrants, perfumes, etc, and mixtures thereof. By way of example and non-exhaustively, such an acceptable adjuvant in the fields of application covered by the present invention, present in the formula in a content of between 0.01 and 20% by weight relative to the total weight of the composition, preparation and other supplement mentioned above, is cited in particular in the “International Cosmetic Ingredient Dictionary and Handbook” published by the “Personal Care Products Council (PCPC)” of the American Cosmetic Association. Finally, these same compositions mentioned above may comprise, in certain embodiments, at least one additional active ingredient, but with the person skilled in the art taking care that these possible additional active ingredients, as well as their proportions, are chosen in such a way that the recognized beneficial properties in these compositions mentioned above are not, or not substantially, altered by the intended addition. Such additional active ingredients may be chosen, without this list being limitative, from depigmenting agents, photoprotective agents, agents that stimulate the barrier function, moisturizing or humectant agents, desquamating agents, soothing agents, exfoliating agents, agents that stimulate cell proliferation such as fibroblasts and keratinocytes, deglycosylating agents, agents that stimulate collagen or elastin synthesis or prevent their degradation, agents that stimulate glycosaminoglycan or proteoglycan synthesis or prevent their degradation, antioxidants or anti-free radical or anti-pollution agents, agents that stimulate lipolysis, draining or detoxifying agents, anti-inflammatory agents, penetration accelerators, desquamating agents, soothing and/or anti-irritant agents, astringent agents, exfoliating agents, agents acting on microcirculation, etc, and mixtures thereof, such an additional active ingredient being present in the formula in a content of between 0.0001 and 20% by weight, preferably between 0.01 and 5% by weight, relative to the total weight of the above-mentioned composition.

The invention also relates to the non-therapeutic cosmetic or dermocosmetic use of a complex according to the invention as defined above, or of a cosmetic or dermocosmetic composition as defined above, for:
a) correcting non-pathological hyperseborrheic conditions of the skin and/or scalp, preferably the scalp, advantageously to correct scalp irritation related to non-pathological hyperseborrheic conditions of the scalp; and/or
b) promoting hair growth or regrowth; and/or
c) preventing and/or reducing the signs of aging of the skin, preferably selected from a skin exhibiting an alteration in its viscoelastic properties and a skin exhibiting an alteration in the cohesion of its tissues.

As indicated above, the aforementioned non-therapeutic cosmetic or dermocosmetic use is intended for “healthy” skin or “healthy” hair—or the area(s) of healthy skin—as opposed to “unhealthy” skin or hair—or a/the area(s) of unhealthy skin—presenting a skin or hair pathology.

In the case of a topical administration to the skin, this use is carried out on one or more areas of “healthy” skin or on “healthy” hair, i.e. one or more areas of skin or hair that are not affected by a skin or hair pathology.

According to a particular embodiment, particularly in the case of routes of administration other than topical administration to the skin, the aforementioned non-therapeutic cosmetic or dermocosmetic use is carried out on an individual who does not have a skin or hair pathology. In the context of the present invention, this individual is referred to as a “healthy subject,” as opposed to a “sick subject” with a skin or hair pathology.

Another aspect of the invention, distinct from the “cosmetic” or “dermocosmetic” aspect mentioned above, also concerns the “dermatological” aspect. In this respect, the invention also relates to a complex (or a composition comprising or consisting essentially of such a complex) formed between:
a) a heterocyclic nitrogen biomolecule selected from purine nucleobases and/or their methylxanthine alkaloid derivatives, free or in N-glycosylated form; and
b) a silicic acid consisting of orthosilicic acid and/or at least one of its water-soluble oligomers;
   said complex being formed via the establishment of at least one weak chemical bond, preferably at least one hydrogen bond, between at least one hydroxyl group of said silicic acid b) and said heterocyclic nitrogen biomolecule a), preferably at the level of at least one of the nitrogen atoms of said heterocyclic nitrogen biomolecule a); the molar ratio between a) and b) being between about 0.5 and about 15 (for example between 0.5 and 15), for use in the treatment of pathological seborrhoea, acne, rosacea and/or atopic dermatitis.

The invention also relates to the use of a silicic acid b) as defined above for:
i) promoting the bioavailability of a heterocyclic nitrogen biomolecule a) as defined above; and/or
ii) increasing the solubility of a heterocyclic nitrogen biomolecule a) as defined above.

### Definitions.

### General Definitions.

“Nucleic acids and derivatives” must be taken to mean both an organic macromolecule (polymer) consisting of a sequence of nucleotides and its constituent monomeric units (the latter may be free or functionalized by an N-glycosidic bond).

“Orthosilicic acid” must be taken to mean the molecule with the raw structure (H₄SiO₄).

“At least one of its water-soluble oligomers” must be taken to mean one or more of the oligomeric forms as described in the article written by the Japanese team “Igarashi M. and al, Nature communications, 2017, vol. 8(140), pp. 1-8”.

“Complex” or “molecular complex” must be taken to mean a molecular entity formed by a free association between two or more component molecular entities (ionic or uncharged), or the corresponding chemical species, with the bond between the components furthermore normally being weaker than in the case of a covalent bond (Source: IUPAC, 1994, 66, 1077—Glossary of terms used in physical organic chemistry— IUPAC— Recommendations 1994— on page 1098).

“Hydrogen bond” must be taken to mean an attractive interaction between a hydrogen atom in a molecule or molecular fragment X—H where X is more electronegative than H, and an atom or group of atoms in another molecule. A typical hydrogen bond can be represented as X—H••• Y—Z, where the three dots denote the bond and where X—H represents the hydrogen bond donor, the acceptor being an electron-rich region such as a non-bonding doublet on Y. Evidence for hydrogen bond formation may be experimental or theoretical, or a combination of both (Henry M., Interference International Review of Science, 2015, and references cited).

“IUPAC systematic nomenclature” must be taken to mean a name according to a standardized system of chemical compounds based on the application of the strictest recommendations established by the International Union of Pure and Applied Chemistry (“UICPA” in French for “Union Internationale de Chimie Pure et Appliquee”, or “IUPAC” in English), which are recommendations that aim to ensure that a name corresponds to one and only one molecular structure.

“Synergy of action” must be taken to mean that there is a synergistic effect when the combined effect of two chemical products is greater than the sum of the effects of each product taken individually. For example: 2+2>>4. [https://www.cchst.ca/oshanswers/chemicals/synergism.html].

“In compliance with controlled ratio and conditions” must be taken to mean a molar ratio of heterocyclic nitrogen biomolecules limited to that of purine nucleobases or one of its methylxanthine alkaloid derivatives a)/silicic acid b), established after a comparative screening of multiple tests aimed at defining a ratio between these two entities, likely to confer on the complex according to the invention the most favorable possible biological behavior, as well as the identification of operating conditions that allow simultaneous solubilization of the heterocyclic nitrogen biomolecule a) and silicic acid b) entities.

“Non-pathological hyperseborrheic conditions of the skin and/or scalp” must be taken to mean an excessive sebum secretion that gives the skin or scalp a greasy, shiny, or even oily appearance, without being associated with a dermatological pathology.

By “their methylxanthine alkaloid derivatives”, as understood by the person skilled in the art, there are meant compounds of the xanthine type, methylated and primarily originating from the secondary metabolism of plants and their purine nucleobases (Monteiro J. P. et al., Molecules, 2016, vol. 21, 974; Ashihara H. et al., Plant Nucleotide Metabolism—Biosynthesis, Degradation, and Alkaloid Formation, 2020, pp. 213-229).

### DETAILED DESCRIPTION

The detailed description below is intended to explain the invention in a sufficiently clear and complete manner, notably using examples, but should in no way be regarded as limiting the scope of protection to the particular embodiments and examples presented hereafter.

### EXAMPLES

Example 1: Three examples of formulations of compositions according to the invention, for cosmetic or dermocosmetic use, all of them containing a complex according to the aforementioned invention, are mentioned below:

TABLE 1
Formula A (cream)

| | |
|---|---|
| Complex according to the invention [ortho-silicic | 5% |
| acid/adenine] | |
| Capric/caprylic triglycerides | 5% |
| Hemisqualane | 3% |
| C14-22 alcohols (and) C12-20 alkyl glucosides | 1.7% |
| Polyacrylate 6-crosspolymer | 0.5% |
| Glycerin | 2% |
| 4-hydroxyacetophenone | 0.5% |
| Glyceryl caprylate | 0.75% |
| Water | q.s. to |
| | 100% |

TABLE 2
Formula B (scalp serum)

| | |
|---|---|
| Complex according to the invention [ortho-silicic acid/ | 5% |
| 9-b-D-ribofuranosyladenine] | |
| 4-hydroxyacetophenone | 0.5% |
| Erythritol | 3% |
| Trimethyl-[2-(2-methylprop-2-enoyloxy)ethyl]azanium | 0.5% |
| chloride | |
| 2,4-dihydroxy-N-(3-hydroxypropyl)-3,3- | 0.3% |
| dimethylbutanamide | |
| Denatured alcohol | 5% |
| Glyceryl caprylate | 0.75% |
| Water | q.s. to |
| | 100% |

TABLE 3
Formula C (hair lotion)

| Complex according to the invention [ortho-silicic acid/ | % |
|---|---|
| 1,3,7-trimethylxanthine] | |
| Sodium benzoate | 0.7% |
| Glycerin (vegetable) | 3% |
| Xanthane gum | 0.05% |
| Butylene glycol | 2% |
| Phenoxyethanol | 0.5% |
| Water | qsp |
| | 100% |

Furthermore regarding the aforementioned formula B, it should be noted that its use in an in vivo study conducted on humans (clinical trial, carried out under confidentiality) has yielded very favorable preliminary results (vs. placebo) on the scalp, both at the level of its seborrheic condition (decrease of sebum production) and its hydration (increase).

Other in vivo studies conducted on humans (clinical trials, conducted under confidentiality) and with the aforementioned formula B are also ongoing, to illustrate the use of the present invention, specifically with a focus on the following parameters (vs. placebo):
reduction in the number of hairs that fall out when brushing
reduction in the number of hairs pulled out upon traction
increase in density
increase in the number of hairs in the anagen phase
reduction in the number of hairs in the telogen phase.

Example 2: The invention is illustrated below, purely on an indicative basis, by the following tests mentioned above in the description of the invention (tests 1 to 5)

Test 1: demonstrating the ability of the complex according to the invention to limit the secretion of lipids produced and accumulated in and by sebocytes

Experimentally, the test is performed on a model of immortalized human sebocyte cell lines, modified to express a functional androgen receptor (ref Bioalternatives “SEB066662AR” in Qima Life Sciences study report No. SD230106). Specifically, these “SEB06662” sebocytes are seeded in a 96-well plate and cultured for 24 hours in a culture medium. This medium is then replaced by the test medium containing, or (in the case of the control) not containing, the reference “cerulenin” (a natural inhibitor of fatty acid biosynthesis and steroid metabolism) or the active ingredient to be tested. After a further 4 hours of incubation, a lipogenic stimulant mixture (a mixture of vitamin C, vitamin D3, insulin, calcium, and dihydrotestosterone “DHT”) is then added and the cells are incubated again for 7 days. On D+3, half of the medium is removed and the treatments (including the lipogenic mixture) are renewed. Unstimulated control conditions are carried out in parallel. On D+7, the cells are rinsed, fixed, and permeabilized. The lipid droplets contained in the cells are then labeled using a specific fluorescent lipid probe (Bodipy®) and subjected to image analysis using software (INCell Analyzer™ 2200—GE Healthcare). Ten photos are taken per well (20× magnification). The labelling is quantified by measuring the fluorescence intensity normalized to the total number of cells (mean %). All experimental conditions are performed in triplicate (n=3). The results, in particular expressed as a percentage of accumulated lipids, are collated in Table 4 below. They were obtained for the following different configurations:

```
    - case 1, namely:
      . the “control”, i.e. without the addition of an active ingredient;
    - case 2, namely:
      . the reference inhibitor (cerulenin);
    - case 3, namely:
  . the complex according to the invention, in solution [batch No. E18127], combining (hydrogen
bonds), on the one hand, the compound “9-β-D-ribofuranosyladenine” (as the heterocyclic
nitrogen biomolecule a)) and, on the other hand, ortho-silicic acid and/or at least one of its water-
soluble oligomers (as silicic acid b), ortho-silicic acid being the starting silicic entity), in a molar
ratio (a/b) of 2.28.
```

TABLE 4

| Compound | quantity | inhibition (%) of |
|---|---|---|
| (concentration) | (%) of lipids | lipid formation |
| Case 1 [control] | 100 | N/A |
| Case 2 [reference] | 54 | −46 |
| (10 μM) | | |
| Case 3 [according to the invention] | 78 | −22 |
| (0.3%) | | |

The results underline that the complex according to the invention has an ability to significantly inhibit the formation of lipid droplets induced by a lipogenic mixture containing androgens, i.e. an ability to reduce lipid synthesis and storage in sebocytes, which is of particular interest for correcting non-pathological hyperseborrheic conditions of the skin and/or scalp.

Test 2: demonstrating the ability of the complex according to the invention to significantly stimulate the proliferation of cells in the dermal papilla of the human hair follicle

Experimentally, the test is performed on human dermal papilla cells from human follicles (abbreviated “HFDPC” for “Human Follicle Dermal Papilla Cells”) obtained from a temporal facelift on a 66-year-old Caucasian patient (supplied by Promocell: reference C-12071-batch 485Z013). The cells are then cultured at 37° C. and 5% CO₂ in a culture medium (“Human Follicle Dermal Papilla Cell Growth Medium+supplement Mix”—supplied by Promocell: reference C-26501). On D0. these HFDPCs are seeded in 24-well plates at a density of 1.10⁴ cells/cm². On D+1, the HFDPCs are then treated in 500 μl of “HFDPC Growth Medium” culture medium in the presence of the compounds to be tested. On D+2, the effects of the treatments on cell proliferation are evaluated using a test frequently used in HFDPC cells, namely the “MTT test” or “3-(4,5-dimethylthiazol-2-yl)-2,5-diphenyltetrazolium bromide (MTT) test.” The culture medium is replaced with 500 μL of a 0.5 g/L MTT solution. Then after 3 hours of incubation, the formazan generated is dissolved in 500 μL of DMSO. The absorbances are then measured at 570 nm using the “Biotech EPOCH2 spectrophotometer (Agilent)”. Cell proliferation is finally expressed as a % of DO₅₇₀ₙₘ relative to the average DO₅₇₀ₙₘ of the untreated controls. The results, collating the average values obtained from three independent experiments, are presented in Table 5 below. They were obtained for the following different configurations:

```
- case 1, namely:
  . the “control”, i.e. without the addition of an active ingredient;
- case 2, namely:
    . the compound “9-β-D-ribofuranosyladenine” [batch No. E18427], tested alone;
- case 3, namely:
  . ortho-silicic acid and/or at least one of its water-soluble oligomers [batch No.
E18428], tested alone;
- case 4, namely:
    . the complex according to the invention, in solution [batch No. 5148.23],
combining (hydrogen bonds), on the one hand, the compound “9-β-D-
ribofuranosyladenine” (as the heterocyclic nitrogen biomolecule a)) (case 3) and, on the
other hand, ortho-silicic acid and/or at least one of its water-soluble oligomers (as silicic
acid b), ortho-silicic acid being the starting silicic entity) (case 4), in a molar ratio (a/b) of
2.28.
```

TABLE 5

| | cell | increase (%) |
|---|---|---|
| Compound | proliferation | proliferation |
| (concentration) | (%) | of HFDPC |
| Case 1 [control] | 100 | N/A |
| Case 2 | 99 | −1% |
| (0.25%) | | |
| Case 3 | 105 | +5% |
| (0.25%) | | |
| Case 4 [according to the invention] | 113 | +13% |
| (0.25%) | | |

The results underline that, compared to its two constituent elements taken separately, the complex according to the invention, moreover even through an objectified phenomenon of synergistic action, has a significantly greater ability to stimulate the proliferation of cells in the dermal papilla of the human hair follicle, which is of particular interest in promoting hair growth or regrowth.

Test 3: demonstrating the ability of the complex according to the invention to significantly limit hyperprolferation of keratinocytes induced by inflammatory stress

Experimentally, the test is performed on a line of immortalised keratinocyte cells “HaCaT” (supplier: ATCC), maintained by subculturing in complete “DMEM” culture medium (containing 1 g/l glucose supplemented with 10% foetal calf serum) in a humid atmosphere at 37° C. and 5% CO₂. On D0, HaCaT cells are seeded in 24-well plates at a density of 2.10⁴ cells/cm². On D+1 and D+2, the cells are treated in complete DMEM culture medium containing 0.5% foetal calf serum, with the compounds to be tested and in the presence of inflammatory stress generated by the addition (15 ng/ml) of bacterial lipopolysaccharides (LPS) responsible for a hyperproliferation reaction in keratinocytes (“LPS stress”). After two 24-hour treatment periods, the cells are detached, recovered and counted using a Scepter Millipore counting pipette, and the values are presented as the average number of cells. The results, collating the average values obtained from three independent experiments, are presented in Table 6 below. They were obtained for the following different configurations:

```
- case 1, namely:
  . the “control”, i.e. without the addition of an active ingredient and without LPS-stress;
- case 2, namely:
  . the “LPS control”, i.e. without the addition of an active ingredient and with LPS stress;
- case 3, namely (with LPS stress):
    . the complex, in solution [batch No. 5120.22], combining the compounds
“methylsilanetriol”, “(3-hydroxypropoxy)-(methyl)silanediol” and “9-β-D-
ribofuranosyladenine” in a molar ratio of 1, compounds such as methylsilanetriol or (3-
hydroxypropoxy)- (methyl)silane diol being some forms of “organic silicon″ which the
applicant possesses and which are known to exhibit significantly a superior behavior in
water compared to that of orthosilicic acid, as well as excellent properties in terms of
assimilation and stability;
- case 4, namely (with LPS stress):
  . the complex according to the invention, in solution [batch No. E18127], combining
(hydrogen bonds), on the one hand, the compound “9-β-D-ribofuranosyladenine” (as the
heterocyclic nitrogen biomolecule a)) and, on the other hand, the ortho-silicic acid and/or
at least one of its water-soluble oligomers (as silicic acid b), ortho-silicic acid being the
starting silicic entity), in a molar ratio (a/b) of 2.28.
```

TABLE 6

| Compound | quantity of | stimulation (%) of |
|---|---|---|
| (concentration) | cells/cm² | proliferation |
| Case 1 [control] | 143,388 | N/A |
| Case 2 | 171,813 | +20% |
| Case 3 | 152,025 | +6% |
| (0.5%) | | |
| Case 4 [according to the invention] | 130,438 | −9% |
| (0.5%) | | |

The results underline that the complex according to the invention has an ability to significantly reduce the hyperproliferation of keratinocytes induced by inflammatory stress.

Test 4: demonstrating the ability of the complex according to the invention to improve the viscoelasticity of the skin

Experimentally, the test is performed on skin explants from abdominoplasties of 55-year-olds (Biomeca reference: 23031PA) and 61-year-olds (reference: 23032PA), preserved in a “DMEM-antibiotics” survival medium. Several “punches”, i.e. skin discs comprising the epidermis and dermis, of the same size (1.20 cm in diameter), are placed on an insert at the air/liquid interface in a survival medium (Gibco DMEM+antibiotic cocktail, Biomeca reference: 31966021). The skin samples are then placed in a controlled temperature and humidity environment during the experiment (relative humidity: >93% at 37° C.).

The compounds to be tested are applied topically over the entire surface of the punch samples (100 μl applied to the center of the punch samples), twice a day for 11% days according to a defined protocol (Biomeca study report, ref. DEV-2023-0495). The “Atomic Force Microscopy (AFM)” acquisitions, consisting of force-volume (FV) acquisition at the skin level in an air environment, are performed directly on the skin explants and obtained using an atomic force microscope (Bioscope Resolve—Bruker) to which an epifluorescence microscope is attached (Leica DMi8). Such a combination also allows correlative images to be acquired between mechanical and fluorescent acquisitions. The force-volume (FV) acquisitions obtained correspond to a multitude of measurement points, from which the viscoelastic properties, expressed as “viscoelasticity (UA)”, are ultimately extracted. The results, combining the average values obtained from four independent experiments, are collated in Table 7. They were obtained for the following different configurations:

```
- case 1, namely:
  . the “control”, i.e. without the addition of an active ingredient;
- case 2, namely:
    . the complex, in solution [batch No. 5120], combining the compounds
“methylsilanetriol”, “(3-hydroxypropoxy)-(methyl)silanediol” and “9-β-D-
ribofuranosyladenine” (case 2) in a molar ratio of 1, compounds such as methylsilanetriol
or (3-hydroxypropoxy)-(methyl)silane diol being some forms of “organic silicon” which
the applicant possesses and which are known to exhibit significantly a superior behavior in
water compared to that of orthosilicic acid, as well as excellent properties in terms of
assimilation and stability;
- case 3, namely:
    . the complex according to the invention, in solution [batch No. 18127], combining
(hydrogen bonds), on the one hand, the compound “9-β-D-ribofuranosyladenine” (as the
heterocyclic nitrogen biomolecule a)) and, on the other hand, ortho-silicic acid and/or at
least one of its water-soluble oligomers (as silicic acid b), ortho-silicic acid being the
starting silicic entity), in a molar ratio (a/b) of 2.28.
```

TABLE 7

| | Global | Increase (%) in |
|---|---|---|
| Compound | viscoelasticity | viscoelasticity |
| (concentration) | (UA) | (UA) |
| Case 1 [control] | 1 | N/A |
| Case 2 | 1.16 | +16% |
| (5%) | | |
| Case 3 [according to | 1.89 | +89% |
| the invention] | | |
| (5%) | | |

The results underline that the complex according to the invention has an ability to significantly improve the viscoelastic properties of the skin, which is particularly useful for improving skin hydration and barrier function, as well as for correcting the formation of wrinkles and skin slackening.

Test 5: process for preparing a complex according to the invention formed between, on the one hand, 9-β-D-ribofuranosyladenine (as the heterocyclic nitrogen biomolecule a)) and, on the other hand, a silicic acid consisting of orthosilicic acid and/or at least one of its water-soluble oligomers (as silicic acid b), orthosilicic acid being the starting silicic entity)

In order to obtain 100 g of the complex according to the invention, 9-β-D-ribofuranosyladenine (8.7 g; 32.5 mmoles) is first solubilized in a mixture composed of demineralized water (40 g) and 1,3-dihydroxypropane (50 g). The pH of the solution is then acidified with an inorganic and/or organic acid to a pH of 3. Then, under mechanical stirring and at room temperature, 0.44 g (14.2 mmoles) of a concentrated silicate solution (solution at 90 g/kg silicon) obtained from rice husks (a by-product of the cereal industry containing 9% silicon) is added to the aforementioned solution of 9-β-D-ribofuranosyladenine, while ensuring that the pH of the mixture is maintained between 3 and 3.5 (again using an inorganic and/or organic acid). After adding demineralized water (up to q.s. for 100) and then filtering, a clear and colorless (complex) solution is obtained.

1. A complex formed between:
a) a heterocyclic nitrogen biomolecule selected from purine nucleobases and/or their methylxanthine alkaloid derivatives, free or in N-glycosylated form; and
b) a silicic acid consisting of orthosilicic acid and/or at least one of its water-soluble oligomers;
said complex being formed via the establishment of at least one weak chemical bond between at least one hydroxyl group of said silicic acid b) and said heterocyclic nitrogen biomolecule a);
the molar ratio between a) and b) being between about 0.5 and about 15.

2. The complex according to claim 1, wherein said heterocyclic nitrogen biomolecule is a free purine nucleobase.

3. The complex according to claim 1, wherein said heterocyclic nitrogen biomolecule is an N-glycosylated purine nucleobase.

4. The complex according to claim 3, wherein said N-glycosylated purine nucleobase is selected from 9-β-D-ribofuranosyladenine and/or 9-β-D-ribofuranosylguanine.

5. The complex according to claim 1, wherein said heterocyclic nitrogen biomolecule is a methylxanthine alkaloid derived from a purine nucleobase, selected from 1,3,7-trimethylxanthine, 1,3-dimethylxanthine, 3,7-dimethylxanthine and/or 1,3-dimethylxanthine-7-acetic acid, preferably consisting of 1,3,7-trimethylxanthine.

6. The complex according to claim 1, wherein said silicic acid is orthosilicic acid.

7. The complex according to claim 1, wherein said water-soluble oligomers of orthosilicic acid are disilicic acid, linear trisilicic acid and cyclic trisilicic acid, preferably disilicic acid.

8. The complex according to claim 1, wherein said silicic acid b) is derived from silicon-containing plant biomass.

9. The complex according to claim 8, wherein said silicic acid is selected from the grass species Oryza sativa (rice), Panicum miliaceum (millet), Triticum spp. (wheat), Zea mays (maize), Hordeum vulgare (barley), Avena sativa (oats), Secale cereale (rye), Sorghum bicolor (sorghum), Triticum dicoccum (spelt), Triticum turanicum (Khorasan wheat), Eragrostis tef (teff), Digitaria exilis (fonio), and the pseudo-cereals Chenopodium quinoa (quinoa), Amaranthus spp. (amaranth), Fagopyrum esculentum (buckwheat).

10. A composition comprising at least one complex formed between:
a) a heterocyclic nitrogen biomolecule selected from purine nucleobases and/or their methylxanthine alkaloid derivatives, free or in N-glycosylated form; and
b) a silicic acid consisting of orthosilicic acid and/or at least one of its water-soluble oligomers;
said complex being formed via the establishment of at least one weak chemical bond between at least one hydroxyl group of said silicic acid b) and said heterocyclic nitrogen biomolecule a),
the molar ratio between a) and b) being between about 0.5 and about 15, wherein:
i) the molar ratio between said heterocyclic nitrogen biomolecule a) and said silicic acid b) is between about 0.5 and about 15, and/or
ii) the pH of said composition is between about 2.5 and about 4.5.

11. The composition according to claim 10, characterised in that said composition further comprises at least one cosmetically or dermocosmetically acceptable excipient.

12. The composition according to claim 11, wherein said cosmetic or dermocosmetic composition furthermore being in a form suitable for topical administration to the skin and said at least one excipient furthermore being physiologically acceptable to the skin.

13. The composition according claim 11, characterised in that said composition further comprises at least one additional active ingredient selected from depigmenting agents, photoprotective agents, agents that stimulate the barrier function, moisturizing or humectant agents, desquamating agents, soothing agents, exfoliating agents, agents that stimulate cell proliferation such as fibroblasts and keratinocytes, deglycosylating agents, agents that stimulate collagen or elastin synthesis or prevent their degradation, agents that stimulate glycosaminoglycan or proteoglycan synthesis or prevent their degradation, antioxidants or anti-free radical or anti-pollution agents, agents that stimulate lipolysis, draining or detoxifying agents, anti-inflammatory agents, penetration accelerators, desquamating agents, soothing and/or anti-irritant agents, astringent agents, agents acting on microcirculation, and mixtures thereof.

14. A non-therapeutic cosmetic or dermocosmetic use of the composition according to claim 10, for:
i) correcting non-pathological hyperseborrheic conditions of the skin and/or scalp; and/or
ii) promoting hair growth or regrowth; and/or
iii) preventing and/or reducing the signs of aging of the skin, preferably selected from a skin exhibiting an alteration in its viscoelastic properties and a skin exhibiting an alteration in the cohesion of its tissues.

15. The composition according to claim 10, for use in the treatment of pathological seborrhoea, acne, rosacea and/or atopic dermatitis.

16. A use of a silicic acid consisting of orthosilicic acid and/or at least one of its water-soluble oligomers for:
i) promoting the bioavailability of a heterocyclic nitrogen biomolecule selected from purine nucleobases and/or their methylxanthine alkaloid derivatives, free or in N-glycosylated form; and/or
ii) increasing the solubility of a heterocyclic nitrogen biomolecule selected from purine nucleobases and/or their methylxanthine alkaloid derivatives, free or in N-glycosylated form.
